(19) United States
(12) Patent Application Publication
(10) Pub. No.: US 2008/0311625 A1
(43) Pub. Date: Dec. 18, 2008

(54) Immortal Pluripotent Stem Cell Line, Cell Lines Derived Therefrom, Methods of Preparing Thereof and Their Uses

(76) Inventors: Luisa Gennero, Torino (IT); Antonio Ponzetto, Moncalieri Torino (IT); Andrea Savarino, Vinovo Torino (IT)

Correspondence Address: Steinfl & Bruno, 301 N Lake Ave Ste 810, Pasadena, CA, 91101, US

(21) Appl. No.: 12/094,147

(22) PCT Filed: Nov. 16, 2006
(86) PCT No.: PCT/IB2006/054288

§ 371 Date: May 16, 2008

(30) Foreign Application Priority Data
Nov. 18, 2005 (IT) TO2005A000819

(51) Int. Cl. C12P 21/04; C12N 5/08

(52) U.S. Cl. 435/70.1; 435/366; 435/368; 435/370; 435/371; 435/372

(57) ABSTRACT

The present invention relates to immortal pluripotent stem cells derived from a human leukaemia cell line, preferably a human monocytoid cell line and more preferably the human monocytoid cell line, THP1. The present invention further relates to cell lines derived from the immortal pluripotent stem cell line having the phenotype of cell strains characteristic of human tissues, particularly having a human hepatocyte phenotype, as well as the methods for preparing thereof. The present invention further relates to the use of the derived cell line with a human hepatocytic phenotype for the production of albumin and blood coagulation factors.

The present invention relates to immortal, pluripotent stem cells (PSC-THP1) derived from a leukaemia cell line, particularly the THP1 monocytoid cell line. Furthermore, the present invention relates to cell lines derived from the pluripotent stem cell line PSC-THP1, having the phenotype of cell strains which are characteristic of human tissues, particularly having a human hepatocytic phenotype. Furthermore, the present invention concerns methods for obtaining a pluripotent stem cell line, and the cell lines derived therefrom having specific phenotypes, as well as the use of the cell line having a human hepatocytic phenotype derived from the pluripotent stem cell line PSC-THP1 for the production of albumin and blood coagulation factors.

### BACKGROUND OF THE INVENTION

Stem cells are fundamentally important for all living organisms and are characterised by two peculiarities which distinguish them from other cell types. Firstly, they constitute a continuous and inexhaustible source of non-specialised cells capable of replicating. Secondly, stem cells have the ability to become specialised and can differentiate into different cell types in response to localised signals. Said event occurs under the circumstances where a damaged body tissue so requires. Under certain physiological or experimental conditions, stem cells are induced to change and take on specialised functions (for example muscle myocytes or liver hepatocytes etc.). Thus, stem cells have the ability to multiply and also change so as to give rise to many different types of cells, and ultimately have the potential ability to replace damaged tissues [1-15]. A commonly accepted definition of a “stem cell” is that of a cell with two characteristics [2]:

1) the ability for unlimited or prolonged self-regeneration, i.e. the ability for long-term self-reproduction without differentiating;
2) the ability to give rise to transient progenitor cells, with limited proliferative capacity, which can give rise to highly differentiated cells (for example hepatic, nervous, muscular, haematic cells etc.).

The ability of stem cells to differentiate into specific tissues varies, depending on the origin of the cells, and the developmental stage of the organism from which they are extracted.

Thus, there are several types of stem cells [2]:

1. Totipotent stem cells, with the ability to differentiate into all the cell lines necessary to form an embryo, including the cells which give rise to the placenta and the surrounding membranes.
2. Pluripotent stem cells, with the potential to differentiate into any adult animal cell type, but not totipotent cells capable of giving rise to an embryo.
3. Multipotent stem cells, with the ability to multiply and be maintained in culture, but without the ability for limitless regeneration; furthermore, these cells are capable of giving rise to a limited range of cell types.

Pluripotent stem cells may be obtained from blastocyst stage embryos. Embryonic stem cells have a great ability to proliferate and differentiate into an enormous number of different cell types, including bipotent hepatic oval cells and hepatocytes. Embryonic stem cells are the most promising for the large-scale production of hepatocytes. However, just as with foetal cells, their usefulness is limited by ethical considerations, difficulty of isolation, the possibility of rejection and the risk of inducing tumour development.

However, pluripotent stem cells have also been identified in adult humans, in tissues such as the bone marrow, liver, synovia, dental pulp, heart, intestine, peripheral blood, etc.

Studies carried out in recent years have allowed us to understand how to recognise pluripotent stem cells, how to select them, how to culture them and how to induce them to form various adult cell types by means of growth factors and other regulatory proteins. It is sufficient to mention that, in humans, the bone marrow stem cells, from which other blood cell lines are formed, have the molecules CD14+ and/or CD29+ and/or CD34+ and/or CD44+ and/or CD45+ and/or CD71+ and/or CD90+ and/or CD105+ and/or CD117+ as recognition markers, and when purified, they are capable of reconstituting the entire population of blood cells in patients who have received ablative doses of radiation and chemotherapy, and they can do this at a speed proportional to the quantity of cells used [1].

It is know that for transplanting stem cells (CD14+ and/or CD29+ and/or CD34+ and/or CD44+ and/or CD45+ and/or CD71+ and/or CD90+ and/or CD105+ and/or CD117+ haematopoietic progenitors) derived from umbilical cord or other sources, compatibility between donor and recipient is essential. For this reason, a number of bone marrow and placental blood banks have been established. In the case of an allogenic transplant, the search for a compatible donor may take a rather long time; on the other hand, in the case of an autologous transplant, i.e. stem cells from the same transplant patient (from marrow or peripheral blood), the stem cells must first be removed, isolated, cultured, expanded and stored intact, even for quite some time. Indeed, progenitor stem cell (CD14+ and/or CD29+ and/or CD34+ and/or CD44+ and/or CD45+ and/or CD71+ and/or CD90+ and/or CD105+ and/or CD117+) viability has important effects on the ability of the transplant to take [1-2].

In adult humans, various tissues including bone marrow, peripheral blood, liver, intestine, dental pulp, synovia, etc., contain stem cells capable of differentiating into adult hepatocytes. The therapeutic use of such cells is very interesting, given the ease with which they can be cultured (ex vivo). Since they are derived from the patient, they are always compatible, and have no associated ethical problems [1].

Adult bone marrow contains hematopoietic (from blood) and stromal (which form the scaffolding of organs) progenitor cells. Among the hematopoietic cell lines, over the long term, the so-called “HSC” cells give rise to various cell lines, including endothelial cell lines. Mesenchymal stromal cells have the same capability [2].

Adult blood contains hematopoietic progenitor stem cells. According to the work by Huberman (2003), particular pluripotent “HSC” (CD14+ and CD34+) cells can be identified among hematopoietic cells, which when suitably stimulated using growth factors, can differentiate and specialise into cells typical of a number of body parts, including the liver [1-2].

In 2003, Huberman et al. [1] identified a group of cells (CD14+ and/or CD34+) present in the human peripheral blood monocytic fraction, with stem cell-like and pluripotent characteristics (PSCs, Pluripotent Stem Cells). These cells can be induced to acquire macrophagic, lymphocytic, epithelial, endothelial, neuronal and hepatocytic phenotypes without the need to fuse with pre-existing mature tissue.

However, such cells have the disadvantage that, when grown in culture medium enriched with growth factors, within 30-40 days after isolation in vitro, they become senescent and die. In the case where they are grown in medium without growth factors, the above phenomena are accelerated: within 7-8 days following isolation in vitro they become senescent and die.

Therefore, the pluripotent circulating CD14+/CD34+ monocytic stem cells described by Huberman et al. [1] and in the international patent application WO2006044842, may be defined as “mortal”, i.e. not immortal. [1]. Furthermore, studies on cell death, carried out by the present inventors, according to the method of Huberman et al., on the population of PSCs isolated from the peripheral blood of 10 volunteers, have revealed the increased importance of the protein p27 (a nuclear protein which controls apoptosis) at the thirtieth day in cells treated with growth factors, and at the seventh day in cells treated with growth factor free media. Said data would seem to confirm that progressive cellular senescence mediated “programmed cell death” actually exists.

Said observations in relation to death, senescence and cellular apoptosis can also be applied to the cells described in EU patent EP1436381B1, in the EU patent application EP1506999 and in the US patent application US2005/0233447 (Kremer et al.). According to Kremer et al., monocyte derived dedifferentiated human stem cells are characterised by the presence of a monocyte-specific, membrane-associated surface antigen, CD14 and by the presence of at least one pluripotency marker selected from CD117, CD123 and CD135.

The afore-cited patents refer to obtaining monocytes by means of separating human blood, and direct isolation from internal organs if separation from human blood is not possible, such as in the case of anaemia or leukaemia patients. Therefore, on the basis of the content of said patents, it is possible to assume that the cells described are prone, since they are non-tumour cells, to progressive cellular senescence dependent “programmed cell death”.

### SUMMARY OF THE INVENTION

The object of the present invention is that of providing an immortal, pluripotent stem cell line, which is single to acquire, which is stable over time and easily differentiated in order to obtain specialised cell lines to be used for the production of biological molecules.

According to the present invention said object is achieved thanks to the solution specifically claimed in the appended claims. The claims constitute an integral part of the technical teaching provided herein in relation to the invention.

The main claim refers to a pluripotent stem cell line, characterised in that it is derived from a human leukaemia cell line and further characterised in that it is immortal.

The stem cells of the present invention are derived from a leukaemia cell line and are thus immortal cells. By the term “immortal” is meant that the cells, once grown in culture, are capable of essentially infinite replication, without becoming senescent.

One particularly preferred embodiment of the present invention relates to an immortal pluripotent stem cell line derived from a human monocytic leukaemia cell line, preferably the human cell line THP1.

In one particularly preferred embodiment, the immortal pluripotent human stem cell line according to the invention, is the immortal pluripotent stem cell line designated as PSC-THP1, deposited with the Advanced Biotechnology Centre—Interlab Cell Line Collection, Genoa, Italy, under the accession number ICLC PD No. 05005 on 18 Oct. 2005.

In one further embodiment, the invention concerns a method of conditioning and stimulating a human monocytic leukaemia cell line to become an immortal pluripotent stem cell line. More specifically, and in one particular embodiment, said human monocytic leukaemia cell line is the cell line THP1, which is conditioned and stimulated to become the immortal pluripotent stem cell line PSC-THP1 by means of cell biology techniques and using induction with growth factors. In one particularly preferred aspect, the human monocytic leukaemia cell line (e.g. THP1) is conditioned and stimulated to become an immortal pluripotent stem cell line (e.g. PSC-THP1) by culturing in the presence of interleukin 2 (IL-2) and at least one between the macrophage colony stimulating factor (M-CSF), leukaemia inhibitory factor (LIF) and interleukin 6 (IL-6).

The immortal cell line PSC-THP1 constitutes an in vitro model provided with the functions characterising stem cells, and as such, can be used in diagnostic applications, the study prevention and treatment of diseases.

In one further embodiment, the present invention concerns immortal cell lines derived from the immortal pluripotent stem cell line of the invention, said derived cell lines having the phenotype of cell strains characteristic of human tissues, particularly having a human hepatocytic phenotype.

In another preferred embodiment, the present invention relates to a method of inducing the differentiation of an immortal pluripotent stem cell line according to the invention into derived immortal cell lines having the phenotypes of cell strains from human tissues of interest. In particular, the present invention concerns a method of differentiating the immortal PSC-THP1 cell line into a derived immortal cell line having the above two characteristics, namely the hepatocytic phenotype and immortality.

PSC-THP1 induced towards hepatocytic differentiation become hepatocytes (PSC-THP1 liver like cells) and hepatic oval cells, and as such can be used in the diagnosis, study, prevention and treatment of chronic and acute liver diseases. Therefore, in one preferred embodiment, the present invention concerns an immortal cell line having a hepatocytic phenotype derived from the immortal pluripotent stem cell line PSC-THP1, said immortal cell line with a hepatocytic phenotype, being designated as “PSC-THP1 liver like” or “PSC-THP1 hepatocyte like”. This cell line was deposited with the Advanced Biotechnology Centre—Interlab Cell Line Collection, Genoa, Italy under the accession number ICLC PD No. 06003 on 5 Jun. 2006.

In a further embodiment, the present invention concerns the use of the immortal cell line with a hepatocytic phenotype derived from the immortal pluripotent stem cell line PSC-THP1 of the invention, for the production of albumin and blood coagulation factors.

The cell line THP-1 is a known human monocytoid cell line (i.e. leukaemic and immortal), derived from culturing leukaemic monocytes present in the peripheral blood from a one year old Japanese child affected with monocytic leukaemia (Tsuchiya et al.) [16]. Circulating monocytes from this child donor placed in culture did not expire and replicated with high frequency (every two days) in vitro; said monocytes had been made immortal by virtue of their leukaemic origin.

The leukaemic monocytic cells are defined as “monocytoid”, i.e. with phenotypic characteristics similar to, but not identical to, the cells from which they originated, i.e. monocytes. Indeed, monocytes are cells with a precise cellular morphology which undergo cell ageing (biological senescence) and cell death independently from external factors, while the monocytoid cells are immortalised by tumour genes, whereby they never age and have highly variable morphology strictly dependent from the environmental conditions.

An immortal cell population capable of perpetual and infinite replication, giving rise to the cell line THP-1, was isolated from the monocytoids of the Japanese child [16]. Therefore, the appropriate definition for the immortal THP-1 cells is not that of “monocytes” but, since they are leukaemic, that of “monocytoid cells”, having the peculiar characteristic of being highly immature and immortal [1-27].

Therefore, by the term immortal cell line is meant a population of cells which, once placed in culture, are capable of essentially infinite replication, without becoming senescent.

The immortal pluripotent stem cell line PSC-THP1 of the present invention, derived from the immortal cell line THP1, has been obtained by the present inventors inducing the immortal cell line THP1 to dedifferentiate thereby obtaining cells with characteristics typical of pluripotent stem cells, but which advantageously also preserve the characteristic of immortality.

Subsequent conditioning of the immortal cell line PSC-THP1 has allowed the attainment of a cell line which is also immortal with the typical functions of hepatocytes. In particular, these immortal PSC-THP1 cells have been induced to differentiate thereby assuming the morphology and functional characteristics of hepatocytes.

Hepatocytes are the functional cells of the liver which produce the plasma proteins responsible for maintaining homeostasis and coagulation.

Thus, the possibility of inducing differentiation of the immortal PSC-THP1 cells into immortal hepatocytic cells provides a stable and long lasting, continuous cellular system for producing i) albumin with the physico-chemical, biological and physiological characteristics of human albumin, and ii) human blood coagulation factors, thereby becoming a source of choice for the production of such proteins, useful for the treatment of many diseases.

By way of example, with reference to albumin, the following pathologies may be cited: liver failure, hypoalbuminaemia, hypovolemia, nephrosic syndrome, Menetrier's disease, entero-enteric fistulas, burns, hepatitis, hepatic fibrosis, cirrhosis, serious hydrosaline retention in liver cirrhosis or following paracentesis, in malabsorption syndromes.

By way of example, with reference to coagulation factors, the following pathologies may be cited: haemorrhage (for example congenital or acquired alterations of the vascular walls; thrombocytopenia or thrombocytopathy, i.e. platelet deficiencies where the numbers can also be normal; Congenital or acquired deficiencies of one or more of the coagulation factors, i.e.: Factor VIII or IX deficiencies (haemophilia), combined Factor V and VIII deficiencies, Factor XIII deficiency, antiplasmin, increased levels of plasminogen activator and PAI (Plasminogen Activator Inhibitor) deficiency, isolated platelet factor 3 deficiency, dysfibrinogenemia, Von Willebrand's disease, potential Factor XII deficiency, precallicrein and high molecular weight kininogen deficiency; hepatopathies, circulating anticoagulants, oral anticoagulant therapy, heparin, disseminated intravascular coagulation; excessive fibrinolysis mechanism activity), thrombosis (for example, alterations, generally acquired, of the vascular walls; congenital or acquired deficiencies of the natural blood coagulation inhibitors, for example AT III, Protein C or Protein S deficiencies; significant and persistent increases in platelet levels; fibrinolytic mechanism deficiencies).

### DESCRIPTION OF THE INVENTION

Purely by way of non-limiting example, the present invention will now be described in detail with reference to some preferred embodiments.

PSC-THP1 cells have been directed to becoming specialised (in the sense of maturing and differentiating) and become cells with the typical functions of hepatocytes (PSC-THP1 liver like cells). PSC-THP1 cells have been induced to differentiate, assuming the morphology and functional characteristics of hepatocytes by subjecting them in culture to the continual stimulation generated by HGF (Hepatocyte Growth Factor), glucose, a combination of Interleukin 2 and Interleukin 6 and aminoacids. For said purpose, various concentrations of HGF have been tested. At low HGF concentrations (25 and 50 ng/mL), the differentiation of PSCs towards hepatocytic cells is slow and the appearance of numerous oval cells is observed (80% of the cells in culture), some mature hepatocytes (10% of the cells in culture) which produce high concentrations of albumin and some immature hepatocytes or hepatoblasts (10% of the cells in culture) which produce alpha-feto-protein and a small activity, between 3% and 5%, of certain coagulation factors such as Antithrombin III, Factor VIII and Von Willebrand's Factor. At medium doses of HGF (75 and 100 ng/mL), the differentiation of PSCs accelerates as shown by the appearance of numerous mature hepatocytes (80% of the cells in culture) which produce a high concentration of albumin (40-120 g per litre of albumin/5 million cells per ml in culture). The number of oval cells is moderate (15% of the cells in culture), and the number of immature hepatocytes or hepatoblasts is also moderate (5% of the cells in culture) producing alpha-feto-protein. The coagulation factors, the activity of which is between 5% and 80%, become more abundant and are listed in detail in Table 1.

TABLE 1
Coagulation factors

Antithrombin III
Von Willebrand Factor Preproprotein
Ser/Cys Proteinase Inhibitor C
Urokinase Plasminogen Activator Receptor (UPAR Human)
Anticoagulant Slow Form Of Thrombin (THRB Human)
Coagulation Factor V
Coagulation Factor VII
Coagulation Factor VIII
Coagulation Factor XIIIB
Alpha-2-Macroglobulin
Protein C (Inactivator Of Coagulation Factors Va And Viiia)
Protein S (Alpha)
APC (Activated Protein C Receptor)
Alpha-1-Antitrypsin Precursor (Alpha-1 Protease
Inhibitor/Alpha-1-Antiproteinase
Cl Esterase Inhibitor

At high HGF concentrations (150 and 200 ng/mL), the differentiation of PSCs slows down and oval cells represent the majority of the observable cells (70% of the cells in culture), the immature hepatocytes or hepatoblasts are reasonably abundant (25% of the cells in culture) and produce alpha-feto-protein, while mature hepatocytes producing a high concentration of albumin are rare (5% of the cells in culture). Furthermore, at such high HGF concentrations modest activity (between 0% and 5%) of the coagulation factors listed in Table 1 is detected.

### Cell Line

The THP-1 monocytoid cells [16] have been washed 3 times by centrifugation at 160 g for 10 minutes at room temperature in RPMI 1640 medium (Life Technologies, Grand Island, N.Y.) and resuspended in 15 cm plates (Lab-Tek chamber slides, Nunc, Kamstrup, Denmark) at the final concentration of 1×10⁶ cells/ml in RPMI 1640 medium supplemented with: 10% FCS (Celbio, Milan, Italy); 100 units/ml of penicillin; 100 μg/ml streptomycin; 160 mg/L gentamycin (Schering-Plough, Milan, Italy); 2 mM L-glutamine (Life Technologies; growth medium); 50 ng/ml M-CSF (Macrophage Colony-Stimulating growth Factor, Peprotech Inc., America, New Jersey); 1000 units/ml LIF (Leukaemia Inhibitory Factor, Santa Cruz Biotechnology, California, USA); 1000 units/ml IL-2 (human recombinant Interleukin 2); 3 nM phorbol-12-myristate 13-acetate (PMA, (Santa Cruz Biotechnology, California, USA).

Two types of controls have been prepared: a negative control of untreated THP-1 cells (1) and one control of THP-1 cells treated with LIF (Leukaemia Inhibitory Factor) alone (2):

(1) The control THP-1 monocytoid cells [16] have been washed 3 times by centrifugation at 160 g for 10 minutes at room temperature in RPMI 1640 medium (Life Technologies, Grand Island, N.Y.) and resuspended in 15 cm plates (Lab-Tek chamber slides, Nunc, Kamstrup, Denmark) at a final concentration of 1×10⁶ cells/ml in RPMI 1640 medium supplemented with: 10% FCS (Celbio, Milan, Italy); 100 units/ml of penicillin; 100 μg/ml streptomycin; 160 mg/L gentamycin (Schering-Plough, Milan, Italy); 2 mM L-glutamine (Life Technologies; growth medium).
(2) The THP-1 monocytoid cells [16] have been washed 3 times by centrifugation at 160 g for 10 minutes at room temperature in RPMI 1640 medium (Life Technologies, Grand Island, N.Y.) and resuspended in 15 cm plates (Lab-Tek chamber slides, Nunc, Kamstrup, Denmark) at a final concentration of 1×10⁶ cells/ml in RPMI 1640 medium supplemented with: 10% FCS (Celbio, Milan, Italy); 100 units/ml of penicillin; 100 μg/ml streptomycin; 160 mg/L gentamycin (Schering-Plough, Milan, Italy); 2 mM L-glutamine (Life Technologies; growth medium); 1.000 units/ml of LIF (Leukemia Inhibitory Factor, Santa Cruz Biotechnology, America, California).

All samples have been incubated for 15 days in a Heraeus thermostatically controlled incubator at a temperature of 37° C. in an atmosphere containing a constant flow of 8% CO₂ (v/v in air).

Samples of all the cells forming the subject of the study, have been washed 3 times by centrifugation at 160 g for 10 min at 37° C. and have been subjected to cytofluorometric analysis (Epics Profile II, Coulter, Hialeath, Fla.) after staining with, anti-human mouse monoclonal antibodies (Mabs), conjugated to R-phycoerythrin, anti-human CD14 (Santa Cruz Biotechnology, California, USA), anti-human CD34 (Santa Cruz Biotechnology, California, USA), anti-CD45 (Santa Cruz Biotechnology, California, USA), anti-c-Kit (Santa Cruz Biotechnology, California, USA) and anti-c-Met (Santa Cruz Biotechnology, California, USA). The cells tested by cell cytofluorimetry (FACS), are largely positive for all the markers of stem cell expression (CD14+, CD29+, CD34+, CD44+, CD45−/+, CD71+, CD90+, CD105+, CD117+, c-Met).

Upon completion of the incubation period, all the cells show fibroblastoid morphology. The cells detached using 2% lidocaine (Sigma Aldrich, Milan, Italy) in PBS [17, 21] have been washed 3 times by centrifugation at 160 g for 10 minutes at 37° C. in RPMI 1640 (Life Technologies, Grand Island, N.Y.) and have been incubated for a second time in accordance with the following description.

The treated controls, destined to remain undifferentiated pluripotent stem cells (PSC-THP1), have been resuspended at a final concentration of 1×10⁵ cells per ml in 6 well plates (Lab-Tek chamber slides, Nunc, Kamstrup, Denmark) in 0.5 ml per well of final solution composed of RPMI 1640 medium supplemented with: 10% FCS (Celbio, Milan, Italy); 100 units/ml of penicillin; 100 μg/ml streptomycin; 160 mg/L gentamycin (Schering-Plough, Milan, Italy); 2 mM L-glutamine (Life Technologies; growth medium); 50 ng/ml M-CSF (Macrophage Colony-Stimulating growth Factor, Peprotech Inc., America, New Jersey); 1000 units/ml LIF (Leukaemia Inhibitory Factor, Santa Cruz Biotechnology, California, USA); 1000 units/ml IL-2 (human recombinant Interleukin 2); 3 nM phorbol-12-myristate 13-acetate (PMA, Santa Cruz Biotechnology, California, USA).

The cells have been incubated for 23 days in a Heraeus thermostatically controlled incubator at a temperature of 37° C. in an atmosphere containing a constant flow of 8% CO₂ (v/v in air) with 0.25 ml of medium being replaced every 5-7 days.

The samples destined for stimulation to become specialised hepatocytic cells (PSC-THP1 liver like cells) have been resuspended at a final concentration of 1×10⁵ cells per ml in 6 well plates (Lab-Tek chamber slides, Nunc, Kamstrup, Denmark) in 0.5 ml per well of final solution composed of RPMI 1640 medium supplemented with: 300 ml/L Nutrient HAM Mixture F-12 (Gibco, Grand Island, N.Y.); 4 ml/L HANK'S solution (Sigma Aldrich, Milan, Italy); 100 units/ml of penicillin; 100 μg/ml streptomycin; 160 mg/L gentamycin (Schering-Plough, Milan, Italy); 2 mM L-glutamine (Life Technologies; growth medium); 50 ng/ml M-CSF (Macrophage Colony-Stimulating growth Factor, Peprotech Inc., New Jersey, USA); 1000 units/ml LIF (Leukaemia Inhibitory Factor, Santa Cruz Biotechnology, California, USA); 100 μg/L IL-2 (recombinant human Interleukin 2); 50 μg/L IL-6 (recombinant human Interleukin 6); 3 nM phorbol-12-myristate 13-acetate (PMA, Santa Cruz Biotechnology, California, USA); HGF (hepatocyte growth factor, Peprotech Inc., New Jersey, USA), which has been tested at the following concentrations: 25 ng/mL, 50 ng/mL, 75 ng/mL, 100 ng/mL, 150 ng/mL and 200 ng/mL; 5 uL/mL of non-essential aminoacid solution (Sigma Aldrich, Milan, Italy); 10 g/L Glucose (Sigma Aldrich, Milan, Italy); 10⁻⁷M Dexamethazone phosphate (Soldesam 4 mg/ml solution for injections, AB.FARMACOLOGICO MILAN.Srl); 2 mg/L Scopolamine (N-butylbromide hyoscine, Buscopan 20 mg/ml solution for injections, Boehringer Ingelheim Italy S.p.A.); 10% FCS (Celbio, Milan, Italy), or the FCS can be replaced with the substances listed in Table 2:

TABLE 2

| | | |
|---|---|---|
| Insulin (Sigma Aldrich, Milan, Italy) | 10 | mg/L |
| SyntheChol NSO (Sigma Aldrich, Milan, | 4 | ml/L |
| Italy) | | |
| Linoleic acid (Sigma Aldrich, Milan, | 100 | mg/L |
| Italy) | | |
| Transferrin H (Sigma Aldrich, Milan, | 40 | mg/L |
| Italy) | | |
| Sodium selenite (Sigma Aldrich, Milan, | 6.25 | μg/L |
| Italy) | | |

After 23 days in culture, the surnatants of the test cultures are collected and stored at −80° C. for the following laboratory analyses: the concentrations of microalbumin, calcium, alpha-feto-protein, glucose, glycogen and urea.

After 23 days in culture, cell suspensions of all the test samples have been obtained following incubation for 5-8 minutes with 2% lidocaine (Sigma Aldrich, Milan, Italy) in PBS, by pipetting the cell suspension up and down in the well and then harvesting the solution obtained, as described in the references [17, 21].

The cells have been washed three times by centrifugation at 160 g for 10 minutes at 37° C. with unsupplemented RPMI 1640 (Life Technologies, Grand Island, N.Y.).

From the cell pellet obtained, a small portion of the cells have been resuspended in 15 ml tubes (Lab-Tek chamber slides, Nunc, Kamstrup, Denmark) at a final concentration of 5×10⁵ cells/ml for the subsequent phenotypic analyses (Western Blotting, direct immunofluorescence and FACS) and a portion of the cells have been centrifuged at 160 g for 10 minutes at 4° C. in PBS and the cell pellet obtained has been dried completely and immediately frozen at −80° C. for subsequent RNA analysis (PCR).

### Western Blotting

The samples have been subjected to phenotypic analysis by Western Blotting for markers using anti-CD 14 (Santa Cruz Biotechnology, California, USA), anti-CD 29 (Santa Cruz Biotechnology, California, USA), anti-CD 34 (Santa Cruz Biotechnology, California, USA), anti-CD 44 (Santa Cruz Biotechnology, California, USA), anti-CD 45 (Santa Cruz Biotechnology, California, USA), anti-CD 71 (Santa Cruz Biotechnology, California, USA), anti-CD 90 (Santa Cruz Biotechnology, California, USA), anti-CD 105 (Santa Cruz Biotechnology, California, USA), anti CD 117/c-KIT (Santa Cruz Biotechnology, California, USA), anti c-MET (Santa Cruz Biotechnology, California, USA), anti cytokeratin 7 (Santa Cruz Biotechnology, California, USA), anti cytokeratin 8 (Santa Cruz Biotechnology, California, USA), anti cytokeratin 18 (Santa Cruz Biotechnology, California, USA), anti cytokeratin 19 (Santa Cruz Biotechnology, California, USA), anti cytokeratins 7/17 (Santa Cruz Biotechnology, California, USA), anti albumin (Rockland Immunochemicals, Pennsylvania, USA), anti alpha-feto-protein (Monosan Europa, Netherlands). After five washes, the membranes have been incubated with the corresponding secondary antibodies (1:1000) conjugated to horseradish peroxidase (HRP, SantaCruz Biotechnologies Inc., Santa Cruz, Calif., USA) for 1 hour at room temperature, as reported in the following tables.

### Immunofluorescence Protocol

Cells in suspension have been incubated with 0.2 mM MitoTracker Red for 10 minutes at 37° C. After three washes by centrifugation at 160 g for 10 minutes at room temperature in PBS (pH 7.4), the cell pellets have been resuspended in a fixing solution of 4% paraformaldehyde in RPMI 1640 at pH 7.4, 1 hour at room temperature. After three washes in PBS, the cells have been resuspended in a solution of PBS and 0.1% Triton for 1 hour at 4° C. After three washes in PBS, the cells have been seeded onto slide covers and the liquid allowed to evaporate-off in air. The cells have been blocked with 20% normal goat serum for one hour, and then incubated for 30 minutes with the following anti-human monoclonal antibodies: anti-CD 14 (Santa Cruz Biotechnology, California, USA), anti-CD 29 (Santa Cruz Biotechnology, California, USA), anti-CD 34 (Santa Cruz Biotechnology, California, USA), anti-CD 44 (Santa Cruz Biotechnology, California, USA), anti-CD 45 (Santa Cruz Biotechnology, California, USA), anti-CD 71 (Santa Cruz Biotechnology, California, USA), anti-CD 90 (Santa Cruz Biotechnology, California, USA), anti-CD 105 (Santa Cruz Biotechnology, California, USA), anti CD 117/c-KIT (Santa Cruz Biotechnology, California, USA), anti c-MET (Santa Cruz Biotechnology, California, USA), anti cytokeratin 7 (Santa Cruz Biotechnology, California, USA), anti cytokeratin 8 (Santa Cruz Biotechnology, California, USA), anti cytokeratin 18 (Santa Cruz Biotechnology, California, USA), anti cytokeratin 19 (Santa Cruz Biotechnology, California, USA), anti cytokeratins 7/17 (Santa Cruz Biotechnology, California, USA), anti albumin (Rockland Immunochemicals, Pennsylvania, USA), anti alpha-feto-protein (Monosan Europa, Netherlands) conjugated to R-phycoerythrin (PE) or fluorescein isothiocyanate (FITC). Specific controls with the corresponding isotypes have been devised for each monoclonal antibody (Santa Cruz Biotechnology, California, USA). The nuclei have been stained using Hoechst solution (dilution 1:1000). The cover slips, mounted onto slides using moviol have been examined by light microscopy [16-17].

### Flow Cytofluorometric Analysis (FACS)

The cells have been transferred into 50 ml tubes (Lab-Tek chamber slides, Nunc, Kamstrup, Denmark) and washed three times by centrifugation at 160 g for 10 minutes at room temperature in PBS (pH 7.4). The pellets have been resuspended at a final concentration of 5×10⁵ cells/ml in PBS. The cells in suspension have been incubated with 0.2 mM MitoTracker Red for 10 minutes at 37° C. After washing three times with PBS the cells have been fixed for 1 hour in 4% paraformaldehyde in PBS (pH 7.4) at room temperature. After washing three times in PBS, the cells have been resuspended in a solution of PBS and 0.1% Triton for 1 hour at 4° C. After washing three times in PBS, the cells have been resuspended in a blocking solution of 20% normal goat serum for 1 hour. After washing three times in PBS, the samples have been incubated for 30 minutes with the following anti-human monoclonal antibodies: anti-CD 14 (Santa Cruz Biotechnology, California, USA), anti-CD 29 (Santa Cruz Biotechnology, California, USA), anti-CD 34 (Santa Cruz Biotechnology, California, USA), anti-CD 44 (Santa Cruz Biotechnology, California, USA), anti-CD 45 (Santa Cruz Biotechnology, California, USA), anti-CD 71 (Santa Cruz Biotechnology, California, USA), anti-CD 90 (Santa Cruz Biotechnology, California, USA), anti-CD 105 (Santa Cruz Biotechnology, California, USA), anti CD 117/c-KIT (Santa Cruz Biotechnology, California, USA), anti c-MET (Santa Cruz Biotechnology, California, USA), anti cytokeratin 7 (Santa Cruz Biotechnology, California, USA), anti cytokeratin 8 (Santa Cruz Biotechnology, California, USA), anti cytokeratin 18 (Santa Cruz Biotechnology, California, USA), anti cytokeratin 19 (Santa Cruz Biotechnology, California, USA), anti cytokeratins 7/17 (Santa Cruz Biotechnology, California, USA), anti albumin (Rockland Immunochemicals, Pennsylvania, USA), anti alpha-feto-protein (Monosan Europa, Netherlands). All the antibodies used are monoclonal, conjugated to R-phycoerythrin (PE) or Fluorescein-Isothiocyanate (FITC). Specific controls with the corresponding isotypes have been devised for each monoclonal antibody (Santa Cruz Biotechnology, California, USA). The samples have been subjected to quantitative analysis using a laser cytofluorimeter (Epics Profile II, Coulter, Hialeath, F L) at 488 nm and referred to the percentage of fluorescent cells (PFC) as the geometric mean. Threshold values (gates) have been established using control samples labelled with the corresponding isotypes. All values have been analysed with a minimum threshold of 15,000 cells.

### RNA-PCR (RNA-Polymerase Chain Reaction)

RNA Extraction:

RNA extraction has been performed using Tri Reagent solution (Molecular Research Center, Inc.) in accordance with the manufacturers instructions. The cell pellets have been resuspended in 1 ml of Tri Reagent solution per sample and incubated for 5 minutes at room temperature. Following incubation, 100 μl of bromochloropropane have been added and the samples incubated again for 15 minutes at room temperature. After centrifugation at 12000 g for 15 minutes at 4° C., the aqueous phases have been recovered and transferred into Eppendorf tubes (approx. 500 μl), with the addition of 500 μl of isopropanol. The samples have been incubated for 10 minutes at room temperature, then centrifuged at 12000 g for 8 minutes at 4° C. and washed with 1 ml of 75% ethanol, then centrifuged at 7500 g for 5 minutes an finally the supernatant has been removed using a syringe and the pellets have been carefully dried under a fume hood. Finally, the RNA samples have been resuspended in 50 μl of DNAse-free water.

### Spectrophotometric Measurement:

Resuspend in 50 μl of freshly opened, autoclaved MilliQ H₂O, read 5 μl using the spectrophotometer (dilution: 1:100).

### RT-PCR:

Messenger RNAs have been Retro-Transcribed using the Applied Biosystems “cDNA Archive Kit” (Applied Biosystems). Reactions have been prepared in a total volume of 50 μl, with 10 μl of sample solution+H₂O in the presence of 0.25 IU of RNAse (Promega). The samples have been incubated at 25° C. for 10 minutes and then at 37° C. for 2 hours.

TABLE 3

| | | |
|---|---|---|
| Sample + H₂0 | 10 | μl |
| 10x buffer | 5 | μl |
| 25x dNTPs | 2 | μ1 |
| 10x random primers | 5 | μl |
| multi scribe RT | 25 | μl |
| RNAse | 0.25 | U |

The samples have been introduced into the PCR machine for 10 minutes at 25° C. and 2 hours at 37° C. The cDNAs have been stored at −20° C.

### Removal of Genomic DNA:

Genomic DNA has been removed using the “TURBO DNA-free” system (Ambion), by incubation for 30 minutes at 37° C. The DNAs has been removed by incubating each sample with 5 μl DNAs removing agent, for 2 minutes a room temperature. The samples have then been centrifuged at 10000×g for 1 minute, and finally, 1 μl of each reverse transcribed sample used for Real Time PCR amplification.

TABLE 4

| | |
|---|---|
| RNA | 50 μl |
| DNAse buffer | 5 μl |
| DNAse | 2 μl |

The Real Time PCR amplification has been performed using the IQSYBR Green Supermix 1000×50 mL system (1708884-BioRad) with primers for albumin, alpha-feto-protein, c-Met and GADPH from Sigma. Amplification reactions have been performed using an “ABI Prism 7700 Sequence Detector” (Perkin Elmer). The data have been analysed using the Sequence Detection 1.9.1 software (Perkin Elmer).

### Alpha-Feto-Protein, Urea, Albumin and Microalbumin Assays in the Cell Culture Supernatants Tested

All measurements have been performed using a Modular Analytics P automated system (Roche-Hitachi).

### Identification of Coagulation Factors in the Cell Culture Supernatants Tested

All measurements have been performed using a MALDI-TOF-MS (Micro MX, Waters, Manchester, UK) automated system. The peptides identified have been processed using the ProFound-Peptide Mapping on-line search engine for the reconstruction of the sequences and the identification of the corresponding proteins using the PMF, Peptide Mass Fingerprinting technique (EMBL-EBI, European Bioinformatic Institute; NCBI, National Center for Biotechnology Information).

### Electrolyte (Na⁺, K⁺, Cl⁻) Assay in the Cell Culture Supernatants Tested

Electrolyte assay have been performed using ISE (ion-sensitive) electrodes, fitted with a membrane with such physico-chemical characteristics as to make them selective for a given ion. The magnitude of the electromotive force or voltage at the membrane of the ISE electrode with respect to a reference electrode, is proportional to the concentration of the selected ion in the test sample.

### Glucose Assay in the Tested Cell Culture Supernatants

The supernatant is reacted with Reactive R1 (buffer/ATP/NADP) and Reactive R2 (HK/G-6-PDH). In the presence of such reagents, the reaction of phosphorylation of glucose to G-6-P due to the hexokinase (HK) and in the presence of ATP occur; the G-6-P formed is oxidised by the enzyme G-6-PDH with the consumption of NADP. Measurement of the rate of formation of NADPH, determined photometrically, is proportional to the concentration of glucose contained in the tested sample.

### EXAMPLE 1

### Light Microscopy

### Incubation for 7 Days

After seven days of incubation with RPMI 1640 containing 1000 units/ml of LIF, 50 ng/ml M-CSF and 3 nM phorbol-12-myristate 13-acetate (PMA), the samples revealed the morphological transformation of the THP1 cells from rounded to an elongated fibroblastoid shape adhering to the culture plates. The controls showed a purely rounded shape and were completely in suspension.

### EXAMPLE 2

### Light Microscopy

### Incubation for 23 Days

After 23 days of incubation with RPMI 1640, supplemented as described previously, containing 1000 units/ml of LIF, 50 ng/ml of M-CSF, 50, 75, 100, 150, 200 ng/ml HGF and 3 nM phorbol-12-myristate 13-acetate (PMA), the samples revealed the morphological transformation of the THP1 cells from rounded to tetrahedral shape adhering to the culture plates, positive for the production of: albumin, alpha-feto-protein, glycogen, cytokeratins 7, 8, 17, 18 and 19, OV-6, c-Met receptor (FACS, Immunohistochemistry, WB, PCR) and some coagulation factors (Western Blot, MALDI-TOF MS, 2D electrophoresis), as listed in Table 1. The controls showed only a rounded shape and were all in suspension and negative for albumin, alpha-feto-protein, glycogen, cytokeratins 7, 8, 17, 18 and 19, OV-6.

### EXAMPLE 3

### Characterisation of the THP-1 cells vs. PSCs-THP Cells and PSCs-THP1 Liver Like Cells

The results pertaining to the expression of the surface antigen CD14+, CD29+, CD34+, CD44+, CD45−/+, CD71+, CD90+, CD105+, CD117+, c-Met, cytokeratin 7, cytokeratin 8, cytokeratin 18, cytokeratin 19, cytokeratins 7/17 have been expressed on a quantitative scale, as shown in Table 5.

TABLE 5

| | | | PSC-THP1 |
|---|---|---|---|
| | | | liver |
| Surface | | | like |
| antigens | THP-1 | PSC-THP1 | cells |
| CD14 | ++ | +++ | + |
| CD29 | + | ++++ | −−−−− |
| CD34 | +++ | ++++ | −−−−− |
| CD44 | ++ | ++++ | −−−−− |
| CD45 | ++++ | ++ | −−−−− |
| CD71 | ++ | ++++ | +++ |
| CD90 | + | ++++ | −−−−− |
| CD105 | ++ | +++ | −−−−− |
| CD117 | ++ | +++ | + |
| c-MET | +++ | ++++ | ++ |
| Cytokeratin 7 | + | ++ | +++++ |
| Cytokeratin 8 | + | ++ | ++++ |
| Cytokeratin 18 | ++ | +++ | +++ |
| Cytokeratin 19 | ++ | +++ | +++++ |
| Cytokeratins | ++ | ++ | ++++ |
| 7/17 | | | |

−−−−− = absence of fluorescence
+ = 1-5 fluorescent cells per optical field
++ = 6-10 fluorescent cells per optical field
+++ = 10-20 fluorescent cells per optical field
++++ = 20-50 fluorescent cells per optical field
+++++ > 50 fluorescent cells per optical field

### EXAMPLE 4

### Western Blotting

The cells have been subjected to phenotypic analysis by Western Blotting for the markers albumin and alpha-feto-protein, as illustrated below.

The quantitative indications reported in the following tables should be read according to the following keys:
−−−=no band
−/+=slight presence of a band
+=thin band
++=medium band
+++=broad band
++++=high band
+++++=spreaded band
   1. ALBUMIN (Rockland Immunochemicals, Pennsylvania, USA) The results (Table 6) show negativity for human albumin in the sample of RPMI medium, slight positivity for the production of human albumin in the THP-1 control cells, thin positivity in the PSC-THP1 cells, in contrast to marked positivity for the production of human albumin in the HepG2 cells and abundant positivity in the PSC-THP1 liver like cells and in the human albumin.

TABLE 6
Anti human albumin monoclonal antibody

| RPMI | | | PSC- | PSC THP1 liver | human |
|---|---|---|---|---|---|
| medium | HepG2 | THP-1 | THP1 | like cells | albumin |
| −−− | ++++ | −/+ | + | +++++ | +++++ |

2. ALPHA-FETO-PROTEIN (Monosan Europa, Netherlands), Staining with anti alpha-feto-protein HRP Mabs results follow in Table 7.

TABLE 7
Anti human alpha-feto-protein monoclonal antibody

| | | PSC-THP1 liver | |
|---|---|---|---|
| RPMI | THP-1 | like cells | |
| medium | control | (HGF = 100 ng/mL) | HepG2 |
| −−− | −−− | +++ | +++++ |

After 48 hours (day 2) incubation after stimulation, the following samples have been tested: RPMI medium, THP-1 controls, PSC-THP1 liver like cells, i.e. sample as (HGF, hepatocyte growth factor, 100 ng/ml, since this is the optimal dose) and HepG2 cells (positive control hepatoblastoid cell line). In particular, the results show that the production of human alpha-feto-protein in the sample of RPMI medium and in the THP-1 control cells is negative, with marked positivity in the PSC-THP1 liver like cells and abundant positivity in the HepG2 cells (positive control) as shown in Table 7.

The following samples have been tested: RPMI medium, HepG2 cells (positive control hepatoblastoid cell line), THP-1 controls, PSC-THP1, i.e. a sample as per Example 1, PSC-THP1 liver like cells, i.e. a sample at a concentration of 25 ng/ml HGF (hepatocyte growth factor) after 4, 18, 24, 72, 96, 120 and 196 hours from stimulation. In particular, the results are negative for the production of human alpha-feto-protein in the sample of RPMI medium and in the THP-1 control cells, abundantly positive in the HepG2 cells (positive control) below, and markedly positive in the PSC-THP1 liver like cells with a peak after 18 hours from incubation with the HGF growth factor at a concentration of 25 ng/mL, as summarised in Tables 8 and 9.

TABLE 8
Anti human alpha-feto-protein monoclonal antibody
PSC-THP1 liver-like cells (HGF = 25 ng/mL)

| 4 h | 18 h | 24 h | 72 h | 96 h | 120 h | 196 h |
|---|---|---|---|---|---|---|
| ++ | +++++ | +++ | +++ | +++ | ++ | + |

TABLE 9
Anti human alpha-feto-protein monoclonal antibody
controls (Example 3 + RPMI medium or
negative control, + HepG2 or positive control)

| RPMI medium | THP-1 | PSC-THP1 | HepG2 |
|---|---|---|---|
| −−− | −−− | ++ | +++++ |

The following samples have been tested: RPMI medium, HepG2 cells (positive control hepatoblastoid cell line), THP-1 controls, PSC-THP1, i.e. a sample as per Example 1, PSC-THP1 liver like cells, i.e. a sample at a concentration of 50 ng/ml HGF (hepatocyte growth factor) after 4, 18, 24, 72, 96, 120 and 196 hours from stimulation. In particular, the results are negative for the production of human alpha-feto-protein in the sample of RPMI medium and in the THP-1 control cells, abundantly positive in the HepG2 cells (positive control) below, and markedly positive in the PSC-THP1 liver like cells with a peak between 24 and 72 hours from incubation with the HGF growth factor at a concentration of 50 ng/mL, as summarised in Tables 10 and 11.

TABLE 10
Anti human alpha-feto-protein monoclonal antibody
PSC-THP1 liver-like cells (HGF = 50 ng/mL)

| 4 | 18 | 24 | 72 | 96 | 120 | 196 |
|---|---|---|---|---|---|---|
| hours | hours | hours | hours | hours | hours | hours |
| ++ | +++ | ++++ | ++++ | +++ | ++ | + |

TABLE 11
Anti human alpha-feto-protein monoclonal antibody
controls (Example 3 + RPMI medium or
negative control, + HepG2 or positive control)

| RPMI medium | THP-1 | PSC-THP1 | HepG2 |
|---|---|---|---|
| −−− | −−− | ++ | +++++ |

The following samples have been tested: RPMI medium, HepG2 cells (positive control hepatoblastoid cell line), THP-1 controls, PSC-THP1, i.e. a sample as per Example 1, PSC-THP1 liver like cells, i.e. a sample at a concentration of 100 ng/ml HGF (hepatocyte growth factor) after 4, 18, 24, 72, 96, 120 and 196 hours from stimulation.

In particular, the results are negative for the production of human alpha-feto-protein in the sample of RPMI medium and in the THP-1 control cells, abundantly positive in the HepG2 cells (positive control) below, and markedly positive in the PSC-THP1 liver like cells with a peak after 24 hours from incubation with HGF at a concentration of 100 ng/mL, as summarised in Tables 12 and 13.

TABLE 12
Anti human alpha-feto-protein monoclonal antibody
PSC-THP1 liver-like cells (HGF = 100 ng/mL)

| 4 | 18 | 24 | 72 | 96 | 120 | 196 |
|---|---|---|---|---|---|---|
| hours | hours | hours | hours | hours | hours | hours |
| ++ | ++ | ++++ | +++ | +++ | ++ | + |

TABLE 13
Anti human alpha-feto-protein monoclonal antibody

| RPMI medium | THP-1 | PSC-THP1 | HepG2 |
|---|---|---|---|
| −−− | −−− | ++ | +++++ |

The following samples have been tested: RPMI medium, HepG2 cells (positive control hepatoblastoid cell line), THP-1 controls, PSC-THP1, i.e. a sample as per Example 1, PSC-THP1 liver like cells, i.e. a sample at a concentration of 200 ng/ml HGF (hepatocyte growth factor) after 4, 18, 24, 72, 96, 120 and 196 hours from stimulation. In particular, the results are negative for the production of human alpha-feto-protein in the sample of RPMI medium and in the THP-1 control cells, and abundantly positive in the HepG2 cells (positive control) below, and markedly positive in the PSC-THP1 liver like cells with a peak between 24 and 72 hours from incubation with HGF at a concentration of 200 ng/mL, as summarised in Tables 14 and 15.

TABLE 14
Anti human alpha-feto-protein monoclonal antibody
PSC-THP1 liver-like cells (HGF = 200 ng/mL)

| 4 | 18 | 24 | 72 | 96 | 120 | 196 |
|---|---|---|---|---|---|---|
| hours | hours | hours | hours | hours | hours | hours |
| ++ | ++ | ++++ | ++++ | +++ | ++ | + |

TABLE 15
Anti human alpha-feto-protein monoclonal antibody
controls (Example 3 + RPMI medium or negative
control, + HepG2 or positive control)

| RPMI medium | THP-1 | PSC-THP1 | HepG2 |
|---|---|---|---|
| −−− | −−− | ++ | +++++ |

### EXAMPLE 5

### Stimulation with HGF, HGF Receptor Activating Antibody and Peptide

The growth factor HGF binds to the Met receptor; the HGF receptor binding or activating monoclonal antibodies, or the HGF receptor binding or activating peptides, are agonists of the Met receptor too, and as such can induce the same effects as the HGF natural ligand. This concept is supported by the international patent application PCT/EP2004/05097 entitled “Anti-HGF-R antibodies and the use thereof”.

Hence, various concentrations of HGF have been tested. At low HGF concentrations (25 and 50 ng/mL), the differentiation of PSCs-THP1 towards hepatocytic cells is slow and the appearance of numerous oval cells is observed (80% of the cells present in culture) some mature hepatocytes (10% of the dells in culture) which produce a high concentration of albumin and some immature hepatocytes or hepatoblasts (10% of the cells in culture) which produce alpha-feto-protein.

At medium doses of HGF (75 and 100 ng/mL), the differentiation of PSCs accelerates as shown by the appearance of numerous mature hepatocytes (80% of the cells in culture) which produce a high concentration of albumin (90 g per litre of albumin/5 million cells per ml in culture). The number of oval cells is moderate (15% of the cells in culture), and the number of immature hepatocytes or hepatoblasts producing alpha-feto-protein is also moderate (5% of the cells in culture).

At high HGF concentrations (150 and 200 ng/mL), the differentiation of PSCs slows down and oval cells represent the vast majority of the observable cells (70% of the cells in culture), the immature hepatocytes or hepatoblasts are reasonably abundant (25% of the cells in culture) and produce alpha-feto-protein, while mature hepatocytes producing a high concentration of albumin are rare (5% of the cells in culture).

### EXAMPLE 6

### Characterisation of the THP-1 Cells in Comparison to PSCs-THP Cells and PSCs-THP1 Liver Like Cells by Cytofluorimetry (FACS)

Adult blood contains hematopoietic progenitor cells. Among the hematopoietic cells, the so-called “HSC” cells (CD14+ and CD34+) are pluripotent cells (Zhao, 2003) which, when suitably stimulated using growth factors, can differentiate and specialise into cells typical of a number of body parts, including the liver [1-2].

THP-1 cells in culture incubated with 50 ng/mL MCSF show minimal expression at the seventh day and stabilise on the fifteenth day, when they show widespread and marked stable fluorescence for CD14+, CD29+, CD34+, CD44+, CD45−/+, CD71+, CD90+, CD105+, CD117+, c-Met (PSCs-THP1); after the fifteenth day said fluorescence is maintained stably in culture. Untreated controls show fluorescence for CD29+, CD34+, CD44+, CD90+, CD105+.

From the fifteenth day THP-1 cells in culture incubated with 25, 50, 100, 200 ng/mL HGF show no fluorescence for CD14+, CD29+, CD34+, CD44+, CD45−/+, CD90+, CD105+ (PSCs-THP1), but are slightly positive for c-Met and CD117 and show high fluorescence for albumin, alpha-feto-protein and cytokeratins (7, 17, 8, 18 and 19) and CD71. Untreated controls show fluorescence for CD14 and CD45. The results are reported in Tables 16 and 17 as the geometric mean+/−the Standard Deviation (SD).

TABLE 16

| | | | | PSC-THP1 |
|---|---|---|---|---|
| Surface | THP-1 | THP1-LIF | PSC-THP1 | liver-like |
| antigens | ctrl 1 | ctrl 2 | ctrl 3 | cells sample |
| CD14 | 142 +/− 5 | 183 +/− 15 | 213 +/− 25 | 7 +/− 6 |
| CD29 | 2 +/− 2 | 8 +/− 5 | 20 +/− 8 | 2 +/− 2 |
| CD44 | 3 +/− 1 | 5 +/− 2 | 15 +/− 5 | 9 +/− 6 |
| CD34 | 12 +/− 5 | 16 +/− 9 | 19 +/− 9 | 9 +/− 6 |
| CD45 | 26 +/− 6 | 20 +/− 7 | 19 +/− 4 | 7 +/− 3 |
| CD71 | 23 +/− 1 | 27 +/− 2 | 38 +/− 5 | 59 +/− 30 |
| CD90 | 7 +/− 5 | 16 +/− 9 | 32 +/− 9 | 8 +/− 6 |
| CD105 | 3 +/− 1 | 9 +/− 2 | 28 +/− 7 | 2 +/− 2 |
| CD117 | 36 +/− 2 | 12 +/− 2 | 38 +/− 4 | 4 +/− 2 |
| c-MET | 6 +/− 2 | 10 +/− 4 | 32 +/− 9 | 28 +/− 6 |

TABLE 17

| | | | | PSC-THP1 |
|---|---|---|---|---|
| Antigens | THP-1 | THP1-LIF | PSC-THP-1 | liver like |
| Tested | ctrl 1 | ctrl 2 | ctrl 3 | cells |
| Albumin | 18 +/− 4 | 5 +/− 7 | 18 +/− 10 | 90 +/− 20 |
| alpha-feto-protein | 10 +/− 8 | 5 +/− 2 | 3 +/− 1 | 18 +/− 12 |
| Cytokeratin 7 | 7 +/− 1 | 15 +/− 5 | 19 +/− 3 | 80 +/− 25 |
| Cytokeratin 8 | 5 +/− 2 | 10 +/− 5 | 22 +/− 3 | 85 +/− 53 |
| Cytokeratin 18 | 3 +/− 1 | 7 +/− 2 | 18 +/− 5 | 59 +/− 30 |
| Cytokeratin 19 | 6 +/− 2 | 12 +/− 2 | 25 +/− 5 | 72 +/− 24 |

### Albumin

Samples of cells in culture treated with various concentrations of HGF (most indicative doses 25, 50, 100, 200 ng/mL) incubated for various times (most representative times 7, 15, 23 days) have been analysed.

At low HGF concentrations (25 and 50 ng/mL), the differentiation of PSCs-THP1 towards hepatocytic cells is slow and the appearance of several mature hepatocytes is observed (10% of the cells present in culture) which produce a high concentration of albumin, increasing progressively from day 7 to day 23, and some immature hepatocytes or hepatoblasts (10% of the cells in culture) which produce alpha-feto-protein.

At medium doses of HGF (100 ng/mL), differentiation of the PSCs accelerates as demonstrated by the appearance of numerous mature hepatocytes (80% of the cells in culture) which produce a high concentration of albumin (90 g/L of albumin/5 million cells per mL in culture), again with progressive increase from day 7 to day 23. The number of immature hepatocytes or hepatoblasts producing alpha-feto-protein is moderate (5% of the cells in culture).

At high concentrations of HGF (200 ng/mL), the differentiation of PSCs slows down, the immature hepatocytes or hepatoblasts are reasonably represented (25% of the cells in culture) and produce alpha-feto-protein, while mature hepatocytes producing a high concentration of albumin are rare (5% of the cells in culture).

Concentrations of albumin above the minimum detectable limits are observed in only 2 samples treated with HGF (PSC-THP1 liver like cells) sampled at day 15 to 30 of the experiment. In accordance with the previous analysis, said data indicate the production of albumin by PSC-THP1 liver like cells derived from the pluripotent PSC-THP1 stem cell line.

The data relating to microalbumin assays in the samples tested confirm the previous data on albumin, both numerically and temporally. Infact, concentrations of microalbumin above the minimum detectable limits are observed in only 3 samples treated with HGF (PSC-THP1 liver like cells) sampled at day 7, 15 and 30 of the experiment.

### Untreated THP-1 Control Cells:

At all incubation times, the THP-1 control cells show no significant presence of albumin by cytofluorimetry. By way of example, the data relating to the controls at day 23 are shown.

THP-1 Cells Treated with MCSF (PSCs-THP1):

At all incubation times, the THP-1 cells treated with 50 ng/mL MCSF (PSC-THP1) show no detectable albumin by cytofluorimetry. By way of example, the data relating to the controls at day 23 are shown.

PSCs-THP1 Cells Incubated with HGF at 25 Nanograms/mL (7, 15, 23 days):

At low concentrations of HGF (25 ng/mL and 50 ng/mL), the differentiation of the PSCs-THP1 cells towards hepatocytic cells is slow and the appearance of mature hepatocytic cells is observed (10% of the cells in culture) which produce a high concentration of albumin.

PSCs-THP1 Cells Incubated with HGF at 50 ng/mL (7, 15, 23 Days):

As in the previous example, at reduced concentrations of HGF (25 and 50 ng/mL), the differentiation of the PSCs-THP1 towards hepatocytic cells appears moderate.

PSCs-THP1 Cells Incubated with HGF at 100 ng/mL (7, 15, 23 Days):

At medium doses of HGF (100 ng/mL), the differentiation of PSCs accelerates as shown by the appearance of numerous mature hepatocytes (80% of the cells in culture) which produce a high concentration of albumin (90 g per litre of albumin/5 million cells per ml in culture) again with progressive increase from day 7 to day 23.

PSCs-THP1 Cells Incubated with HGF at 200 ng/mL (7, 15, 23 Days):

At high concentrations of HGF (200, ng/mL), the differentiation of the PSCs slows the mature hepatocytes (5% of the cells in culture) producing a high concentration of albumin.

### Alpha-Feto-Protein

Samples of untreated THP-1 cells in culture (control), treated with 50 ng/mL MCSF (PSC-THP1) and treated with various concentrations of HGF (25, 50, 100, 200 ng/mL) (PSC-THP1 liver like cells) have been analysed at various incubation times (days 7, 15, 23).

At all incubation times, the THP-1 control cells show no production of alpha-feto-protein by cytofluorimetry. By way of example, the data relating to the controls at day 23 are shown.

THP-1 Cells Treated with MCSF (PSCs-THP1):

At all incubation times, the THP-1 cells treated with 50 ng/mL MCSF (PSC-THP1) show no production of alpha-feto-protein by cytofluorimetry. By way of example, the data relating to the controls at day 23 are shown.

THP-1 Cells Treated with HGF (25, 50, 100, 200 ng/mL) (PSC-THP1 Liver Like Cells):

Samples of cells in culture treated with various concentrations of HGF (25, 50, 100, 200 ng/mL) incubated for various times (7, 15, 23 days) (PSC-THP1 liver like cells) have been analysed.

At low concentrations of HGF (25 and 50 ng/mL), the differentiation of the PSCs-THP1 cells towards hepatocytic cells is slow and the appearance of immature hepatocytes or hepatoblasts is observed (10% of the cells in culture) which produce alpha-feto-protein, with maximal expression at day 15 of incubation.

PSCs-THP1 Cells Incubated with HGF at 100 ng/mL (7, 15, 23 Days):

At medium doses of HGF (100 ng/mL), differentiation of the PSCs accelerates as shown by the appearance of a moderate number of immature hepatocytes or hepatoblasts (5% of the cells in culture) producing alpha-feto-protein with progressive increase from day 7 to day 15 of incubation.

PSCs-THP1 Cells Incubated with HGF at 200 ng/mL (7, 15, 23 Days):

At high concentrations of HGF (200 ng/mL), the differentiation of PSCs slows down, the immature hepatocytes or hepatoblasts are reasonably represented (25% of the cells in culture) and produce alpha-feto-protein, with a progressive increase from day 7 to day 15, and a progressive decrease from day 15 to day 23 of incubation.

PSCs-THP1 Cells Incubated with HGF: Expression of Cytokeratins:

The THP-1 cells in culture incubated with 25, 50, 100, 200 ng/mL HGF from the fifteenth day show no strong fluorescence for cytokeratins (7, 17, 8, 18 and 19). All controls and PSC-THP-1 cells show no significant positive signal for alpha-feto-protein and the cytokeratins (7, 17, 8, 18 e 19).

The data relating to alpha-feto-protein assay in the supernatants show no detectable variation in concentration in the tested samples.

### Glucose

The data relating to glucose assays in the supernatants show high concentrations from day 1 to day 7 of the second incubation of the experiment, with an increase in the samples treated with growth factors (MCSF and HGF). Within the time range from day 7 to day 15, a rapid decrease in the concentrations of glucose, first of all in the samples treated with HGF (PSC-THP1 liver like cells) is detected. The data relating to the samples and controls at day 30 of the second incubation show concentrations below the detection limit.

### Electrolytes

The results are summarised in Table 18.

TABLE 18

| Cell | | | | | | | | | | |
|---|---|---|---|---|---|---|---|---|---|---|
| culture | | | | | Micro | | | | | |
| supernatant | AFP | Gluc. | Urea | Alb. | Alb. | Ca⁺⁺ | Mg⁺⁺ | Na⁺ | K⁺ | Cl⁻ |
| assays | ng/mL | mg/dL | mg/dL | g/dL | mg/dL | mEq/L | mEq/L | mEq/L | mEq/L | mEq/L |
| TPH1 g.1 | <0.4 | 66 | 11 | <0.2 | 10.10 | 1.06 | 0.84 | 135 | 6.0 | 105 |
| CTRL | | | | | | | | | | |
| TPH1 M-CSF | <0.4 | 141 | 9 | <0.2 | 8.14 | 1.46 | 0.79 | 143 | 5.8 | 107 |
| d.1 | | | | | | | | | | |
| TPH1 HGF | <0.4 | 95 | 7 | <0.2 | 9.01 | 1.36 | 0.82 | 139 | 5.7 | 113 |
| d.1 | | | | | | | | | | |
| TPH1 d.7 | <0.4 | 109 | 9 | <0.2 | 10.10 | 0.94 | 0.64 | 137 | 6.0 | 104 |
| CTRL | | | | | | | | | | |
| TPH1 M-CSF | <0.4 | 113 | 12 | <0.2 | 7.79 | 1.71 | 0.86 | 142 | 5.5 | 111 |
| d.1 | | | | | | | | | | |
| TPH1 HGF d.7 | <0.4 | <2 | 10 | 0.2 | 180.00 | 2.05 | 0.83 | 156 | 5.8 | 121 |
| TPH1 d.15 | <0.4 | 69 | 10 | <0.2 | 13.00 | 1.04 | 0.65 | 130 | 5.8 | 109 |
| CTRL | | | | | | | | | | |
| TPH1 M-CSF | <0.4 | 66 | 15 | <0.2 | 7.54 | 1.71 | 0.65 | 138 | 5.5 | 113 |
| d.15 | | | | | | | | | | |
| TPH1 HGF | <0.4 | <2 | 11 | 0.5 | 547.00 | 1.88 | 0.76 | 150 | 5.6 | |
| d.15 | | | | | | | | | | |
| TPH1 d.20 | <0.4 | <2 | 12 | <0.2 | 13.60 | 1.14 | 0.76 | 132 | 6.2 | 107 |
| CTRL | | | | | | | | | | |
| TPH1 M-CSF | <0.4 | <2 | 18 | 0.2 | 8.10 | 2.28 | 1.12 | 139 | 5.6 | 113 |
| d.20 | | | | | | | | | | |
| TPH1 HGF | <0.4 | <2 | 10 | 0.9 | 1120.00 | 2.16 | 0.94 | 147 | 5.6 | 114 |
| d.20 | | | | | | | | | | |
| Minimum | 0.4 | 2 | 2 | 5 | 0.2 | 0.23 | 0.10 | 0.06 | 10 | 1.0 |
| detectable | | | | | | | | | | |
| limit | | | | | | | | | | |

### EXAMPLE 7

### Real time PCR

### GAPDH

The data show that the mRNA for GAPDH is uniformly expressed in all tested samples, ensuring the correct storage of the mRNA extracted, and the uniformity in the initial concentrations of the samples examined.

### Albumin

The data show that the presence of the mRNA for albumin is only clear in the HepG2 positive control and in the THP1-PSC liver like cells following stimulation with HGF (optimal treatment range: 50-100 ng/mL HGF) for at least 30 days; in all other controls there appears to be no albumin mRNA.

### Alpha-feto-protein

The data show that the presence of the mRNA for alpha-feto-protein is only clear in the HepG2 positive control, in the 2 THP1-LIF controls, in the THP1-PSC liver like cells following stimulation with HGF (optimal treatment range: 50-100 ng/ml HGF) for at least 10 days (weak expression of the alpha-feto-protein mRNA), at day 15 (maximum peak of expression of the alpha-feto-protein mRNA) and at day 30 (slight expression of the alpha-feto-protein mRNA); in all other controls there appears to be no alpha-feto-protein mRNA.

c-Met

The data show the presence of c-Met mRNA is rather well expressed in all the tested cells. The c-Met mRNA does not appear to be modulated by any treatment.

The results are summarised in Table 19.

TABLE 19

| | Absolute values | | | | Corrected values | | |
|---|---|---|---|---|---|---|---|
| | GAPDH | ALB | AFP | c-Met | ALB | AFP | c-Met |
| HepG2 | 21.46 | 17.22 | 0 | 11.39 | 17.22 | 0 | 11.39 |
| THP1 ctrl | 22.76 | 0 | 0 | 11.29 | 0 | 0 | 10.64 |
| THP1 ctrl | 21.70 | 0 | 1.25 | 11.37 | 0 | 1.24 | 11.24 |
| THP1 ctrl | 21.41 | 0 | 0.83 | 11.16 | 0 | 0.83 | 11.19 |
| THP1 ctrl | 20.55 | 0 | 4.83 | 13.96 | 0 | 5.04 | 14.58 |
| LIF | | | | | | | |
| THP1 10 | 22.12 | 0 | 0.66 | 12.18 | 0 | 0.67 | 12.39 |
| days HGF | | | | | | | |
| THP1 10 | 21.10 | 0 | 0 | 12.76 | 0 | 0 | 12.38 |
| days MCSF | | | | | | | |
| THP1 15 | 20.57 | 0.50 | 4.13 | 13.53 | 0.52 | 4.30 | 14.11 |
| days HGF | | | | | | | |
| THP1 15 | 23.03 | 0 | 0 | 10.98 | 0 | 0 | 10.23 |
| days MCSF | | | | | | | |
| THP1 30 | 21.90 | 8.75 | 1.15 | 8.46 | 8.57 | 1.13 | 8.29 |
| days HGF | | | | | | | |
| THP1 30 | 22.73 | 0 | 0 | 12.72 | 0 | 0 | 12.01 |
| days MCSF | | | | | | | |

### MALDI-TOF-MS

The PSC-THP1 liver like cells culture supernatants have been treated by Western Blotting, and the bands obtained have been cut out and digested. Numerous peptides have been identified from the digestion. The peptides obtained have been processed using the ProFound-Peptide Mapping on-line search engine for reconstruction of the sequences and the identification of the corresponding proteins using the PMF, Peptide Mass Fingerprinting, technique. The production of human albumin and coagulation factors has been confirmed.

### BIBLIOGRAPHY

1. Zhao Y, Glesne D, Huberman E. (2003). A human peripheral blood monocyte-derived subset acts as pluripotent stem cells. Proc. Natl. Acad. Sci. USA. 100(5):2426-31.
2. Stem Cells: Scientific Progress and Future Research Directions. Department of Health and Human Services. June 2001.
3. Uzan G. (2004). Therapeutic use of stem cells. Rev. Prat. 54(13):1399-403.
4. Griffith, L. G. & Naughton, G. (2002). Tissue engineering-current challenges and expanding opportunities. Science. 295, 1009-1014.
5. Wagers, A. J., Christensen, J. L., & Weissman, I. L. (2002). Cell fate determination from stem cells. Gene Ther. 9, 606-612.
6. Lagasse, B., Connors, H., Al Dhalimy, M., Reitsma, M., Dohse, M., Osborne, L., Wang, X., Finegold, M., Weissman, I. L., and Grompe, M. (2000). Purified hematopoietic stem cells can differentiate into hepatocytes in vivo.
7. Theise, N. D., Nimmakayalu, M., Gardner, R., Illei, P. B., Morgan, G., Teperman, L., Henegariu, O., and Krause, D. S. (2000). Liver from bone marrow in humans. Hepatology. 32, 11-16.
8. Osawa, M., Hanada, K., Hamada, H., and Nakauchi, H. (1996). Long-term lymphohematopoietic reconstitution by a single CD34− low/negative hematopoietic stem cell. Science. 273, 242-245.
9. Domen, J. and Weissman, I. L. (1999). Self-renewal, differentiation or death: regulation and manipulation of hematopoietic stem cell fate. Mol. Med. Today. 5, 201-208.
10. Petersen, B. E., Bowen, W. C., Patrene, K. D., Mars, W. M., Sullivan, A. K., Murase, N., Boggs, S. S., Greenberger, J. S., and Goff, J. P. (1999). Bone marrow as a potential source of hepatic oval cells. Science. 284, 1168-1170.
11. Deutsch, G., Jung, J., Zheng, M., Lora, J., and Zaret, K. S. (2001). A bipotential precursor population for pancreas and liver within the embryonic endoderm. Development. 128, 871-881.
12. Crosby, H. A. and Strain, A. J. (2001). Adult liver stem cells: bone marrow, blood, or liver derived? Gut. 48, 153-154.
13. Bianco, P. and Cossu, G. (1999). Uno, nessuno e centomila: searching for the identity of mesodermal progenitors. Exp. Cell Res. 251, 257-263. Exp. Cell Res. 1999 Sep. 15; 251(2):257-63.
14. Roy, V. and Verfaillie, C. M. (1999). Expression and function of cell adhesion molecules on fetal liver, cord blood and bone marrow hematopoietic progenitors: implications for anatomical localization and developmental stage specific regulation of hematopoiesis. Exp. Hematol. 27, 302-312.
15. Korbling, M., Katz, R. L., Khanna, A., Ruifrok, A. C., Rondon, G., Albitar, M., Champlin, R. E., & Estrov, Z. (2002). Hepatocytes and epithelial cells of donor origin in recipients of peripheral-blood stem cells. N. Engl. J. Med. 346, 738-746.
16. Tsuchiya S, Yamabe M, Yamaguchi Y, Kobayashi Y, Konno T, Tada K. Establishment and characterization of a human acute monocytic leukemia cell line (THP-1). (1980). Int. J. Cancer; 26: 171-176.
17. Rabinovitch, M. & De Stefano, M. J. Cell shape changes induced by cationic anesthetics. (1976). J. Exp. Med. 143, 290-304.
18. Nakabo, Y. & Pabst, M. J. Lysis of leukemic cells by human macrophages: inhibition by 4-(2-aminoethyl)-benzenesulphonyl fluoride (AEBSF), a serine protease inhibitor. (1996). J. Leukocyte Biol. 60, 328-336.
19. Vitale, A., Guarini, A., Latagliata, R., Cignetti, A. & Foa, R. Cytotoxic effectors activated by low-dose IL-2 plus IL-12 lyse IL-2-resistant autologous acute myeloid leukaemia blasts. (1998). Br. J. Haematol. April; 101(1):150-7.
20. Semizarov, D., Glesne, D., Laouar, A., Schiebel, K. & Huberman, E. (1998). A lineage-specific protein kinase crucial for myeloid maturation. Proc. Natl. Acad. Sci. USA 95, 15412-15417.
21. Rabinovitch, M. & De Stefano, M. J. (1976) a. Exp. Med. 143, 290-304.
22. Vessey C J, de la Hall P M. Hepatic stem cells: a review. Pathology. 2001 May; 33(2):130-41. Review. PMID: 11358043
23. Zhang Y, Bai X F, Huang C X. Hepatic stem cells: existence and origin. World J. Gastroenterol. 2003 February; 9(2):201-4. Review. PMID: 12532431
24. Petersen B E. Hepatic Oval Cells: “Knocking on Forbidden Doors”. 2000 e-biomed. Vol. 1 Mary Ann Liebert, Inc.
25. Fausto N, Campbell J S. The role of hepatocytes and oval cells in liver regeneration and repopulation. Mech. Dev. 2003 January; 120(1):117-30. Review. PMID: 12490302
26. Overturf K, al-Dhalimy M, Ou C N, Finegold M, Grompe M. Serial transplantation reveals the stem-cell-like regenerative potential of adult mouse hepatocytes. Am. J. Pathol. 1997; 151: 1273-1280
27. Kountouras J, Boura P, Lygidakis N J. Liver regeneration after hepatectomy. Hepatogastroenterology 2001; 48: 556-562.

1. A pluripotent stem cell line derived from the human monocytoid leukaemia cell line THP-1, said pluripotent stem cell line being immortal, CD14+ and being capable of producing a high concentration of albumin upon differentiation with HGF (hepatocyte growth factor) into a derived cell line, the derived cell line having an hepatocytic phenotype, wherein a high concentration of albumin is defined as 40-120 g/l of albumin produced by 5 million cells per ml in culture.

2. The pluripotent stem cell line according to claim 1, wherein the pluripotent stem cell line further expresses on the cell surface at least one of the antigens CD29, CD34, CD44, CD45, CD71, CD90, CD105, CD117, c-Met, cytokeratin 7, cytokeratin 8, cytokeratin 18, cytokeratin 19, cytokeratin 7/17, or any combination thereof.

3. The pluripotent stem cell line according to claim 1, wherein the pluripotent stem cell line is the cell line designated as PSC-THP1 deposited with the Advanced Biotechnology Centre—Interlab Cell Line Collection, Genoa, Italy, under the accession number ICLC PD No. 05005 on 18 Oct. 2005.

4. A derived cell line, said derived cell line being a cell line derived from the pluripotent stem cell line as defined in claim 1, wherein the derived cell line has the phenotype of a cell strain characterising human tissue, said phenotype being selected from lymphocytic, epithelial, endothelial, neuronal and hepatocytic.

5. The derived cell line according to claim 4, wherein said derived cell line possesses a hepatocytic phenotype.

6. The derived cell line according to claim 5, wherein said derived cell line is the cell line designated as PSC-THP1 liver like or hepatocyte like, deposited with the Advanced Biotechnology Centre—Interlab Cell Line Collection, Genoa, Italy, under the accession number ICLC PD No. 06003 on 5 Jun. 2006.

7. A method for obtaining an immortal pluripotent stem cell line according to claim 1, said method comprising culturing a leukaemia cell line in presence of interleukin 2 (IL-2) and at least one factor selected from the group consisting of macrophage colony stimulating factor (M-CSF), leukaemia inhibitor factor (LIF) and interleukin 6 (IL-6).

8. The method according to claim 7, wherein culturing the leukaemia cell line is performed in presence of interleukin 2 (IL-2), macrophage colony stimulating factor (M-CSF) and leukaemia inhibitory factor (LIF).

9. The method according to claim 7, wherein said leukaemia cell line is cultured in the presence of one or more additional substances selected from the group consisting of glucose, amino acids, vitamins, antibiotics, salts, phorbol-12-myristate-13-acetate; dexamethazone, hormones; and any combination thereof.

10. The method according to claim 7, wherein the interleukin 2 is used in an amount comprised between 10 units/ml and 5000 units/ml.

11. The method according to claim 8, wherein the leukaemia inhibitory factor is used in an amount comprised between 10 units/ml and 5000 units/ml.

12. The method according to claim 8, wherein the macrophage colony stimulating factor (M-CSF) is used in an amount comprised between 0.1 and 1000 ng/ml.

13. The method according claim 9, wherein the phorbol-12-myristate-13-acetate is used in an amount comprised between 0.1 nM and 30 nM.

14. The method according to claim 9, wherein the aminoacid is used in an amount comprised between 0.1 μl/ml and 500 μl/ml.

15. The method according to claim 9, wherein the antibiotic penicillin or streptomycin is used in an amount comprised between 0.1 units/ml and 1000 units/ml.

16. The method according to claim 9, wherein the antibiotic gentamycin is used in an amount comprised between 1 mg/L and 1000 mg/L.

17. A method of differentiating an immortal pluripotent stem cell line into a derived cell line according to claim 4, wherein said immortal pluripotent stem cell line is cultured in presence of at least one factor selected from the growth factor specific for said phenotype and a compound capable of activating the receptor for the growth factor specific for said phenotype.

18. The method according to claim 17, wherein said immortal pluripotent stem cell line is cultured additionally in presence of interleukin 2 and at least one factors selected from the macrophage colony stimulating factor, leukaemia inhibitory factor and interleukin 6.

19. The method according to claim 17 or 18, wherein said pluripotent stem cell line is cultured in presence of one or more additional substances as defined in claim 9.

20. The method according to claim 17, wherein said growth factor is selected from the group consisting of endothelial growth factor, macrophage growth factor, lymphocyte growth factor, epithelial growth factor, neuronal growth factor and hepatocyte growth factor.

21. The method according to claim 20, wherein said growth factor is hepatocyte growth factor and wherein said derived cell line has a hepatocytic phenotype.

22. The method according to any of claims 17 to 21, wherein said compound capable of activating the receptor for the growth factor specific for said phenotype is selected from hepatocyte growth factor, hepatocyte growth factor receptor binding or activating peptides, hepatocyte growth factor receptor binding or activating antibodies, hepatocyte growth factor activating proteases, and wherein said derived cell line has a hepatocytic phenotype.

23. The method according to claim 22, wherein said hepatocyte growth factor is used in an amount comprised between 25 ng/ml and 200 ng/ml.

24. The method according to claim 23, wherein said hepatocyte growth factor is used in an amount comprised between 50 ng/ml and 150 ng/ml, preferably between 75 ng/ml and 100 ng/ml, and wherein said derived cell line with a hepatocytic phenotype is constituted by 80% mature hepatocytes, 15% oval liver cells, 5% immature hepatocytes or hepatoblastoids.

25. A method of producing albumin, the method comprising producing albumin from the derived cell line according to claim 5.

26. The method according to claim 25, wherein the derived cell line is cultured in presence of a hepatocyte growth factor, one or more compounds capable of activating the hepatocyte growth factor receptor, one or more hepatocyte growth factor activating proteases, or combinations thereof.

27. The method according to claim 26, wherein the hepatocyte growth factor is used in an amount comprised between 25 ng/ml and 200 ng/ml.

28. The method according to claim 25, wherein the albumin is produced in quantities within the range of 40-250 g/l from a culture at a concentration within the range of 5×10⁵-5×10⁶ cells/ml.

29. A method of producing one or more coagulation factors, the method comprising producing said at least one or more coagulation factors from the derived cell line to claim 5.

30. The method according to claim 9, wherein the amino acid comprises glutamine the antibiotic comprises gentamycin and/or penicillin and/or streptomycin; the salt comprises phosphate or calcium phosphate or sodium bicarbonate, the hormones comprises levothyroxin.

31. The method according to claim 10, wherein the interleukin 2 is used in an amount of 1000 units/ml.

32. The method according to claim 11 wherein the leukaemia inhibitory factor is used in an amount of 1000 units/ml.

33. The method according to claim 12, wherein the macrophage colony stimulating factor (M-CSF) is used in an amount of 25-200 ng/ml.

34. The method according to claim 13, wherein the phorbol-2-myristate-13-acetate is used in an amount of preferably 3 nM.

35. The method according to claim 14, wherein the aminoacid is used in an amount of 5 μl/ml.

36. The method according to claim 15, wherein the antibiotic penicillin or streptomycin is used in an amount of 100 units/ml.

37. The method according to claim 16, wherein the antibiotic gentamycin is used in an amount of 160 mg/L.

38. The method according to claim 23, wherein said hepatocyte growth factor is used in an amount comprised between 50 ng/ml and 150 ng/ml.

39. The method according to claim 23, wherein said hepatocyte growth factor is used in an amount comprised between 75 ng/ml and 100 ng/ml.

40. A method of producing albumin, the method comprising producing albumin from the derived cell line produced by the method according claim 21.

41. The method according to claim 27, wherein said hepatocyte growth factor is used in an amount comprised between 50 ng/ml and 150 ng/ml.

42. The method according to claim 27, wherein said hepatocyte growth factor is used in an amount comprised between 75 ng/ml and 100 ng/ml.

43. A method of producing one or more coagulation factors, the method comprising producing the one or more coagulation factors from the derived cell line produced by the method according claim 21.
